(19) United States
(12) Patent Application Publication
(10) Pub. No.: US 2008/0154243 A1
(43) Pub. Date: Jun. 26, 2008

(54) DELIVERY DEVICE FOR A FLUID

(75) Inventor: John Krumme, Woodside, CA (US)

Correspondence Address: KIRTON AND MCCONKIE, 60 EAST SOUTH TEMPLE,, SUITE 1800, SALT LAKE CITY, UT, 84111, US

(73) Assignee: BETA MICROPUMP PARTNERS LLC, Menlo Park, CA (US)

(21) Appl. No.: 11/613,912

(22) Filed: Dec. 20, 2006

(51) Int. Cl. A61K 9/22

(52) U.S. Cl. 604/892.1

(57) ABSTRACT

A delivery device for a primary fluid comprises a tubular housing which includes a reservoir for the primary fluid, and a chamber for a displacement fluid which extends coaxially with the reservoir inside the housing and is separated from the reservoir by means of a membrane. The device includes an outlet from the reservoir for the primary fluid, and a device for driving the displacement fluid under pressure into the chamber so that the membrane is deformed into the reservoir to cause primary fluid in the reservoir to be displaced towards the outlet.

### BACKGROUND TO THE INVENTION

This invention relates to a delivery device for a fluid.

The flow of fluids through conduits can be controlled using components such as pumps and valves. Pumps and valves can operate to control parameters such as flow rate; adjustment of relative flow rates of constituents in a mixture can be used to vary the composition of the mixture.

Accurate control of flow of a fluid can be important in many medical applications, for example in drug delivery and in the modulation of body fluid drainage. Devices in which flow control is important include pumps for dispensing drugs such as insulin and opiates, and hydrocephalus shunts for drainage of spinal fluids.

Accurate control over the flow of drugs and fluids in medical applications can help to minimise complications in the patient treatment, especially if controlled quantities of drugs can be supplied locally to an affected site. Accurate control can help to optimise efficacy of an administered drug. The use of controlled quantities can also help to minimise wastage of drugs, and therefore to minimise treatment costs. An implanted device for controlling flow of drugs can help to ensure compliance with prescribed drug administration regime by eliminating patient dependence on operation of the device.

Accurate and localised control of a drug can be facilitated by means of implanted control devices. U.S. Pat. No. 6,287,295 relates to an implantable device which relies on a semipermeable membrane to control the rate of drug delivery. However, once implanted, the rate of flow of drug through the membrane cannot readily be adjusted.

It can frequently be required that a delivery device for a drug or other fluid is compact, to facilitate handling when in use. This can be particularly desirable when a delivery device is to be implanted in a patient.

### SUMMARY OF THE INVENTION

The present invention provides a delivery device for a primary fluid which includes a housing providing a reservoir for the primary fluid and a chamber for a displacement fluid, and a device for driving the displacement fluid into the chamber to cause the primary fluid to be displaced from the reservoir.

Accordingly, in one aspect, the invention provides a delivery device for a primary fluid, which comprises:

a. a tubular housing which comprises a reservoir for the primary fluid, and a chamber for a displacement fluid which extends coaxially with the reservoir inside the housing and is separated from the reservoir by means of a membrane,

b. an outlet from the reservoir for the primary fluid,

c. a device for driving displacement fluid under pressure into the chamber so that the membrane is deformed into the reservoir to cause primary fluid in the reservoir to be displaced towards the outlet.

The delivery device of the invention has the advantage that it can be made with a compact shape which can facilitate use, for example when the delivery device is to be carried by a patient or when the delivery device is to be implanted in a patient. This arises from the coaxial arrangement of the reservoir and the chamber within the tubular housing.

It can be preferred for the chamber to be located within the reservoir, for example when the reservoir is annular when viewed in cross-section. Delivery of the displacement fluid to the chamber will then drive the membrane to outwardly into the reservoir, to displace primary fluid from the reservoir. The membrane can be provided by a balloon, which can be inflated by means of the displacement fluid.

When the chamber for the displacement fluid is located within the reservoir, for example when the reservoir is annular when viewed in cross-section along the axis of the device and surrounds the chamber (in a coaxial sense), the primary fluid can be discharged from the reservoir to the reservoir outlet through a conduit. The conduit can extend through the chamber. The conduit can be located on the axis of the device. The chamber for the displacement fluid can itself then have an annular configuration, being defined internally by the external surface of the conduit and externally by the internal surface of membrane. It can then be formed by bonding the membrane to the external surface of the conduit.

It can also be preferred for the reservoir to be located within the chamber, for example in which the chamber is annular when viewed in cross-section along the axis of the device. Delivery of the displacement fluid to the chamber will then drive the membrane to inwardly into the reservoir, to displace primary fluid from the reservoir. This can have the advantage that, when the chamber is annular and surrounds the reservoir (in a coaxial sense), the outlet from the reservoir can be located on or close to the axis of the device.

Preferably, the delivery device includes a source reservoir for the displacement fluid. Preferably, the source reservoir for the displacement fluid is located within the housing. This can mean that the delivery device of the invention can be provided as a single package which contains the fluid which is to be delivered, and the components which are required to deliver the fluid. The volume of the displacement fluid which can be drawn from the source should be no less than the volume of the drug which is to be displaced from the reservoir by means of the displacement fluid. Preferably, the ratio of the volume of the source reservoir for the displacement fluid to the volume of the fluid which is to be delivered is at least about 0.85, more preferably at least about 0.9, especially at least about 1.05, for example about 1.1. Preferably, the value of that ratio is not more than about 1.3, more preferably not more than about 1.2.

Preferably, the source of the displacement fluid is provided by a source reservoir which extends coaxially within the housing with the chamber and the reservoir for the primary fluid. The source reservoir can be arranged so that it is coaxial with and outside the chamber and the reservoir for the primary fluid. The source reservoir can be arranged so that it is coaxial with and inside the chamber and the reservoir for the primary fluid.

Preferably, the driver device is located at one end of the housing.

The driver device can comprise an electro-osmotic device. Electro-osmotic devices apply a potential difference to liquid on opposite sides of a semi-permeable membrane made of a dielectric material. Provided that the liquid is able to yield a high zeta potential with respect to the porous dielectric material of the membrane, the application of the potential difference leads to transmission of charged species, possibly together with solvent (for example which solvates the charged species or as bulk solvent by viscous drag), through the membrane. This technology can be used to control the rate at which a liquid is supplied, for example under pressure which is generated by means of a pump. The technology, including amongst other things details of the materials which can be used for the membrane and as the liquid which is transmitted across the membrane, is discussed in detail in US-A-2002/189947. Subject matter disclosed in that document is incorporated in the specification of the present application by this reference.

WO-2005/021968 discloses a valve which makes use of an electro-osmotic device to control flow of a primary fluid in a primary flow channel. The electro-osmotic device drives a valve fluid to cause a valve member to be displaced between open and closed positions. The capacity for flow of the primary fluid in the primary flow channel is greater when the valve member is in the open position than when it is in the closed position. The valve can be incorporated into a pump when combined with inlet and outlet valves.

The electro-osmotic device can act on the displacement fluid directly when the displacement fluid is able to yield a high zeta potential with respect to the porous dielectric material of the membrane. The application of the potential difference can then lead to transmission of the displacement fluid through the membrane.

The electro-osmotic device can be provided as a pump which can pump the displacement fluid into the chamber without the displacement fluid passing through the porous dielectric material of the membrane. For example, the driver device can include a flow channel for the displacement fluid which is provided by a compressible tube, which can be compressed by the application of pressure caused by a valve fluid passes through a membrane of a porous dielectric material under an applied potential difference. A valve which relies on a compressible tube is disclosed in WO-2005/021968. A pump which makes use of such a valve, in conjunction with an inlet valve and an outlet valve, is also disclosed in that document. The delivery device of the present invention can make use of a device for driving the displacement fluid into the chamber which includes a valve which relies on a compressible tube as disclosed in WO-2005/021968, in conjunction with an inlet valve or an outlet valve or both. The features of relevant valves and pumps that are disclosed in WO-2005/021968 can therefore be incorporated into the delivery device of the present invention and such disclosed subject matter is incorporated in the specification of the present application by this reference.

Factors which might affect the choice of the displacement fluid can include the nature of the device by which it is driven into the chamber. For example, if the displacement fluid is required to pass through the porous dielectric material of the membrane of an electro-osmotic device, it will be required to yield a high zeta potential with respect to the porous dielectric material of the membrane so that it can be transmitted through the membrane on application of a potential difference. The choice of a displacement fluid might also be affected by applicable safety requirements, for example which apply because the delivery device is to be used for a medical application, especially if it is to be implanted in a patient.

The displacement fluid can be a liquid or a gas. It will generally be a liquid when it is required to pass through the porous dielectric material of the membrane of an electro-osmotic device. It will generally then be preferred to include a source reservoir for that liquid. A source reservoir need not be included for certain displacement fluids, for example when the displacement fluid is an ambient fluid to which the delivery device is exposed when in use. For example, when the delivery device is intended to be exposed to atmospheric air, the air can be used as the displacement fluid. When the delivery device is intended to be exposed to a body fluid, especially when the delivery device is implanted in a patient, that body fluid can be used as the displacement fluid. The driver device can then communicate with an opening which is exposed to the ambient fluid to which the exterior of the delivery device is exposed. For example, when the driver device is located at one end of the housing, the opening for ambient fluid can be provided at that end of the housing.

Preferably, the delivery device includes an outlet valve to control flow of the primary fluid through the reservoir outlet. The outlet valve should allow flow of the primary fluid out of the reservoir and restrict (preferably, prevent) flow of fluid in the reverse direction. Suitable constructions of outlet valve for restricting flow of fluid to a single flow direction are known.

A preferred outlet valve can incorporate an electro-osmotic device. A suitable electro-osmotic device can include a flow channel for the primary fluid which is compressible, which is acted on by a working fluid after the working fluid has passed through a membrane of a porous dielectric material. Constructions of suitable outlet valves are disclosed in WO-2005/021968. The outlet valve can be provided in a disk which is located at one end of the delivery device of the invention. When the outlet valve includes a flow channel for the primary fluid, the direction of flow of the primary fluid can be through the disk along its axis, with the working fluid acting in the plane of the disk to compress the flow channel.

The membrane can be capable of deforming resiliently as a result of the supply of the displacement fluid to the chamber. Factors which will affect the selection of a suitable materials for the membrane will include the nature of any materials which the membrane will contact when in use (generally including the primary fluid and the displacement fluid). The membrane can be capable of deforming resiliently as a result of the supply of the displacement fluid to the chamber. Suitable deformable materials might include certain polymers and elastomers.

The membrane can deform as a result of a folded construction, by which its configuration can change on deformation into the reservoir by opening of folds, for example relying on an effect which is similar to that which is referred to as a concertina effect. This has the advantage that expansion of the chamber by means of driven displacement fluid does not require that the deformation forces of an elastomeric material have to be overcome.

Preferably, the cross-section of the housing when viewed along its axis is approximately circular.

Preferably, the tubular housing is elongate so that the ratio of the length of the housing (measured along its axis) to its transverse dimension (which will be its diameter when the housing has a circular cross-section) is at least about 1.0. A delivery device with an elongate housing can be suitable for delivery through a lumen, for example through a catheter or through a lumen within a patient (such as a blood vessel or the alimentary canal). Preferably, the transverse dimension of the housing (which will be its diameter when the housing has a circular cross-section) is not more than about 5 mm, more preferably not more than about 3 mm, especially not more than about 1.5 mm.

Preferably, the cross-section of the housing when viewed along its axis is approximately circular, and the ratio of the length of the housing measured along its axis to its diameter is at least about 1.0, more preferably at least about 1.5, especially at least about 2.0, more preferably at least about 4.0, for example at least about 5.0.

The housing can be squat so that the ratio of the length of the housing (measured along its axis) to its transverse dimension (which will be its diameter when the housing has a circular cross-section) is not more than about 1.0. Accordingly, it can be preferred that Preferably, the cross-section of the housing when viewed along its axis is approximately circular, and the ratio of the length of the housing measured along its axis to its diameter is less than about 1.0, more preferably less than about 0.75, especially not more than about 0.5, more preferably not more than about 0.3, for example not more than about 0.2. A delivery device in which the housing is squat can be suitable for location during use against a surface, for example against the surface of a patient's tissue. Such a delivery device can be implanted sub-cutaneously.

The volume of the reservoir for the primary fluid might be at least about 10 μl. The volume of the reservoir for the primary fluid might be at least about 100 μl. The volume of the reservoir for the primary fluid might be at least about 250 μm. The volume of the reservoir for the primary fluid might be at least about 500 μl. The volume of the reservoir might be not more than about 5000 μl. The volume of the reservoir might be not more than about 2000 μl. The volume of the reservoir might be not more than about 1000 μl.

The material of the housing will be selected according to the physical conditions to which the delivery device will be exposed during use, and to the materials with which the delivery device will come into contact with when in use. The housing might be made from metallic materials for certain applications. When the delivery device is intended to be implanted in a patient, it might be made from materials such as stainless steels and certain titanium alloys. The housing might be made from polymeric materials. Suitable polymeric materials include engineering polymers such as aryl ether ketones (especially polyether-etherketones), polycarbonates, polyurethanes, acetals, and polyolefins, especially certain polyethylenes and certain polypropylenes.

### BRIEF DESCRIPTION OF THE DRAWINGS

FIGS. 1a to 1c are side views and partial isometric views, each partially in section, through a first embodiment of primary fluid delivery device according to the present invention, in full, partially emptied and empty conditions.

FIG. 2 is a schematic sectional elevation through a pump component which is suitable for use in the delivery device shown in FIG. 1.

FIG. 3 is a schematic view of an outlet valve construction which can be used to provide the outlet valve in the delivery device shown in FIG. 1.

FIGS. 4a to 4c are side views, partially in section, through a second embodiment of primary fluid delivery device according to the present invention.

FIG. 5 is a schematic sectional elevation on the line V-V through a pump component which is suitable for use in the delivery device shown in FIG. 4.

FIG. 6 is an enlarged cross-section through a valve which can be used as the driver valve in the pump component which is shown in FIG. 5.

### DESCRIPTION OF PREFERRED EMBODIMENTS

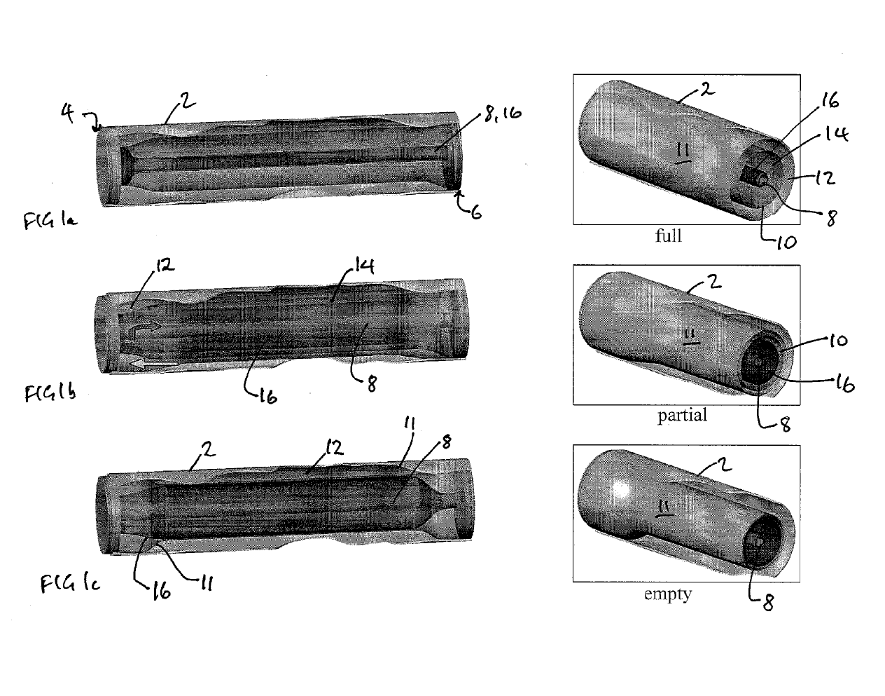

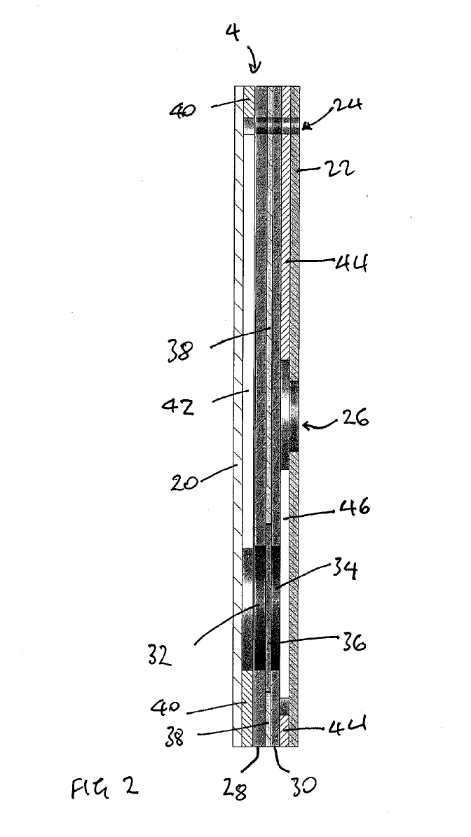

FIGS. 1a to 1c show a delivery device which can be used to deliver a fluid such as a drug in liquid phase. The delivery device comprises a tubular housing having an outer wall 2 which is circular when viewed in cross-section along its length. The diameter of the housing is 1.0 mm, and its length is 10 mm. The thickness of the outer wall of the housing is about 0.05 mm. The housing is formed from a polymeric material such as a polyether-etherketone. The side wall is formed by extrusion, allowing the volume of the housing to be determined by cutting the extrusion to length. The construction of the end walls is discussed below. They are sealed to the side wall by means of an adhesive.

The housing is closed at one end by a pump end wall 4 and at its other end by an outlet end wall 6. A rod 8 extends between the end walls 4, 6 of the housing along the housing axis. The rod is formed from the same polymeric material as that of the housing.

The housing includes a cylindrical inner wall 10 which is spaced apart from the outer wall so as to define an annular space between them. The inner wall is sealed at its ends to the pump end wall and the outlet end wall so that the spaces within the housing, inside and outside the inner wall respectively, are sealed from one another.

The space between the outer wall 2 of the housing and the inner wall 10 contains a first flexible membrane 11. The first flexible membrane defines a source reservoir 12 between it and the inner wall 10 for a displacement fluid.

The space within the inner wall 10 of the housing contains a second flexible membrane 16. The second flexible membrane defines a chamber 14 for a primary fluid such as a drug solution.

The second membrane 16 is located so that the rod 8 is positioned within it. The membrane is sealed to the outer surface of the rod towards the outlet end of the housing. The membrane is sealed to the pump end wall 4 around the rod 8. To facilitate this, the pump end wall can have an axially extending protrusion 18 (see FIG. 1a) so that the membrane can be sealed to a surface which extends generally along the housing axis. The membrane is sealed to the rod and the protrusion by means of an adhesive.

FIG. 2 is a sectional elevation through the pump end wall 4 of the housing, which includes a pump for driving displacement fluid in the source reservoir 12 into the space defined between the membrane 16 and the rod 8 to cause the membrane to expand within the chamber 14 for the primary fluid.

The pump end wall 4 comprises a plurality of layers. The layer 20 which faces outwardly is formed from the same material as the outer cylindrical wall 2 of the housing and extends continuously over the entire end of the housing. The layer 22 which faces inwardly into the housing is formed from the same material. It has a first opening 24 which communicates with the source reservoir 12 for the displacement fluid, and a second opening 26 which communicates with the space defined between the membrane 16 and the rod 8.

First and second intermediate layers 28, 30 contain first and second electrodes 32, 34 which extend over a circular region of the intermediate layers. The diameter of each of the electrodes is 0.8 mm and the thickness of each of them is about 0.15 mm.

A layer 36 of a porous dielectric material is sandwiched between the electrodes 32, 34, consisting of silica. Spacers 38 occupy the space between the first and second intermediate layers which is not occupied by the layer of the porous dielectric material. The thickness of the layer of the porous dielectric material and of the spacers is about 0.15 mm.

Spacers 40 are provided between the outwardly facing layer 20 and the first intermediate layer 28 to define a path 42 for primary fluid to flow between the first opening 24 and the first electrode 32. Spacers 44 are provided between the second intermediate layer 30 and the inwardly facing layer 22 to define a path 46 for primary fluid to flow between the second electrode 34 and the second opening 26.

Appropriate control components for the discharge valve in the discharge end wall of the housing and for the pump in the pump end wall of the housing, including one or more power sources and control circuitry, can be provided within one or more of the housing end walls, for example within or between component layers of one or more of the housing end walls when they are formed as a plurality of layers which are arranged as a laminate.

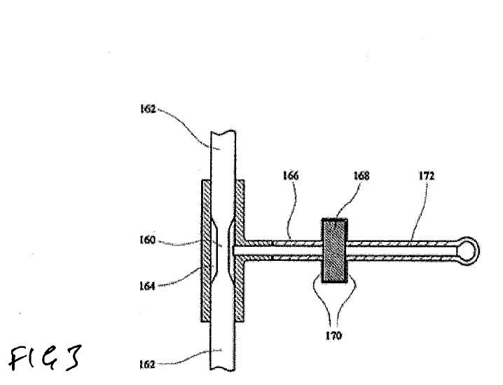

FIG. 3 shows a valve which can be fitted within the outlet end wall 6 of the housing. The valve includes a compressible tube 160 which forms part of the flow channel 162 for the primary fluid. The compressible tube is located within a chamber 164 defined by the housing outlet end wall, which is in fluid communication with the outlet part of the valve fluid channel 166. The valve fluid channel includes a membrane 168 of porous dielectric material, with associated electrodes 170, to cause fluid to flow between the outlet part of the channel and an inlet part 172. The materials of the membrane 168 and the electrodes 70 are the same as the materials used for the pump which is described above with reference to FIG. 2. Accordingly, an increase in fluid pressure in the chamber 168 as a result of flow of valve fluid into the outlet part of the valve fluid channel, due to the application of a potential difference across the membrane 168, can cause compression of the compressible tube, to reduce (or to close completely) the flow of the primary fluid through the compressible tube 160.

Other forms of outlet valve which can be used in the delivery device of the invention will be apparent. A simple form of outlet valve which can be suitable for many applications is a one-way valve, sometimes referred to as a check valve, which allows fluid to flow through it in one direction, and prevents fluid from flowing past it in the opposite direction.

Appropriate control circuitry for the valve in the outlet end wall of the housing and for the pump in the pump end wall of the housing can be provided between the component layers of one or both of the housing end walls. Connections between control circuitry in one end wall and components or other control circuitry in the other end wall can be made through an axially extending conduit within the rod 8.

Operation of the illustrated delivery device can be understood with reference to FIGS. 1a to 1c. The delivery device is supplied initially with a primary fluid in the chamber 14, and with a displacement fluid in the source reservoir 12.

Operation of the pump in the pump end wall 4 of the housing causes displacement fluid to be driven from the source reservoir 12 into the space between the membrane 16 and the rod 8. This causes the membrane to expand within the chamber 14 in which the primary drug fluid is provided.

When the outlet valve in the outlet end wall 6 of the housing is opened, the expansion of the membrane 16 within the chamber 14 causes the drug fluid to be displaced from the chamber and to be discharged from the delivery device.

Preferably, the outlet valve is closed and the pump is deactivated when sufficient drug has been displaced. When the outlet valve includes an electro-osmotic device, it and the delivery device can be operated in reverse to recharge the device.

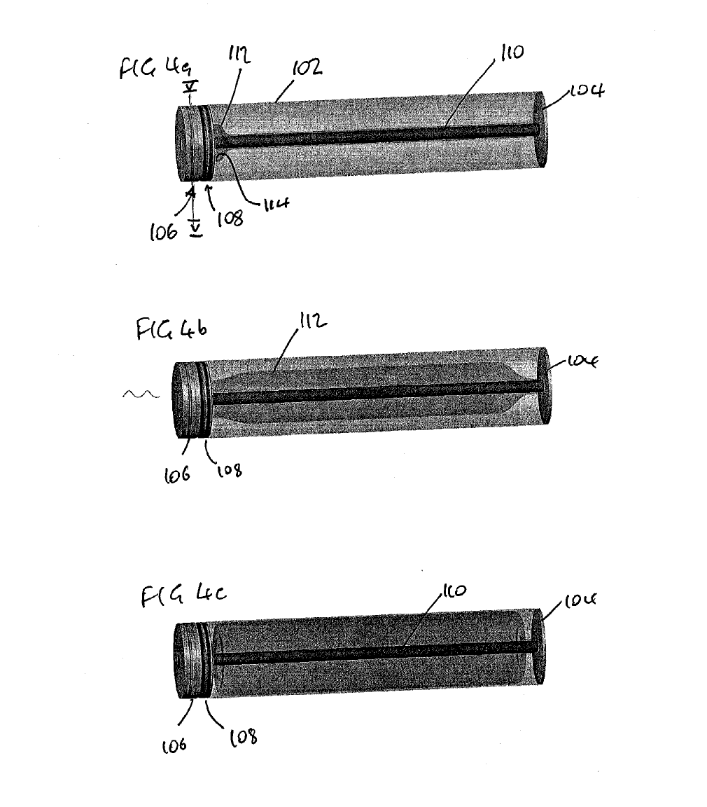

FIG. 4 shows a delivery device which makes use of ambient fluid to displace a drug or other primary fluid from a chamber.

The delivery device comprises a tubular housing having an outer wall 102 which is circular when viewed in cross-section along its length. The diameter of the housing is 1.0 mm, and its length is 10 mm. The housing is formed from a polymeric material such as a poly-propylene. The side wall is formed by extrusion, allowing the volume of the housing to be determined by cutting the extrusion to length. The construction of the end walls is discussed below. They are sealed to the side wall by means of an adhesive.

The housing is closed at the outlet end of the housing with a simple end wall 104 which is provided by a plain sheet of polymeric material.

The housing is closed at its other end by a pair of functional end walls. A first end wall is a pump end wall 106 which is used to drive displacement fluid into the device to cause a primary fluid to be displaced. A second end wall, provided in face to face relationship with the pump end wall 106, is a discharge valve end wall 108. The discharge valve end wall contains a valve which controls the discharge of primary fluid from the delivery device. The discharge valve end wall is sealed to a rod 110 which extends between the discharge valve end wall 108 and the simple end wall 104 at the opposite end of the housing. Primary fluid which is discharged from the delivery device under the control of the discharge valve flows from the discharge valve end wall towards the outlet end of the housing along a conduit within the rod 10 and is discharged through an orifice in the simple end wall 104 at the outlet end of the housing.

A cylindrical membrane 112 is located so that the rod 110 is positioned within it. The membrane is sealed to the outer surface of the rod towards the outlet end of the housing. The membrane is sealed to the discharge valve end wall 108 around the rod 10. To facilitate this, the discharge valve end wall can have an axially extending protrusion 114 (see FIG. 4a) so that the membrane can be sealed to a surface which extends generally along the housing axis. The membrane is sealed to the rod and the protrusion by means of an adhesive.

The space between the membrane 112 and the outer wall 102 of the housing is a reservoir for a primary fluid which is to be delivered using the device. The space within the membrane 112, between the membrane and the surface of the rod 110, can be filled with an ambient fluid which is pumped into that space from outside the device by means of the pump in the pump end wall 106. Such supply of ambient fluid to cause the membrane to expand within the housing causes displacement of the primary fluid from within the reservoir. The displaced fluid is discharged from the device by flowing through the hollow rod 110, through the discharge valve in the discharge valve end wall 108.

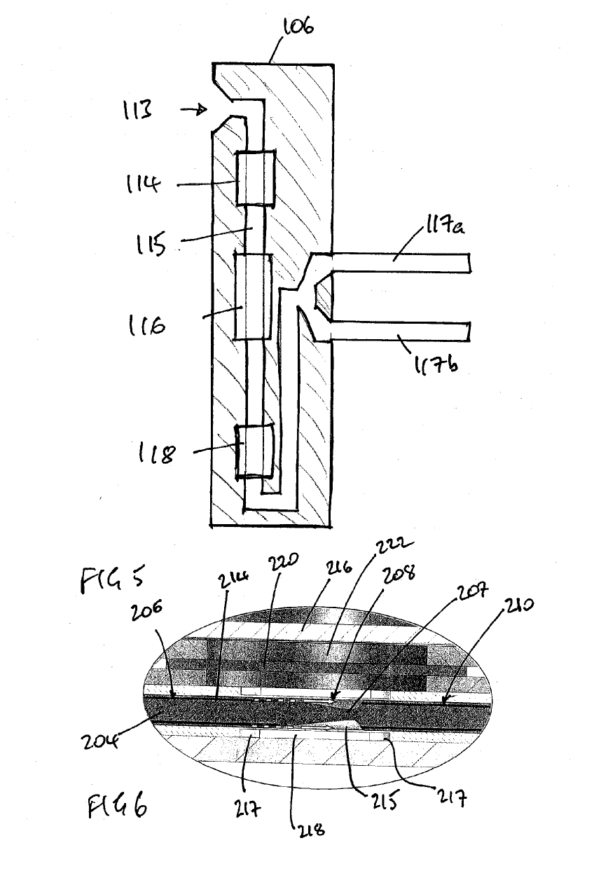

FIG. 5 is a cross-section through a pump end wall which can be used in the delivery device shown in FIG. 4. It makes use of electro-osmotic device to pump ambient fluid to which the device of the invention is exposed when in use into the space between the membrane 112 and the rod 110 to cause the membrane to expand within the chamber for the primary fluid. The pump end wall 106 contains control components for controlling the discharge of primary fluid from the reservoir. The end wall has an inlet 113 for the primary fluid to enter from the reservoir. The end wall includes a passage 115 for the primary fluid to flow through it, from the inlet to an outlet 117 which communicates with the space within the membrane 112, between the membrane and the rod 110, through two outlet branches 117a, 117b, which extend through the discharge valve end wall 108. These include a pump which is made up of an inlet valve 114, a driver valve 116 and an outlet valve 118. Constructions of valve which can be used as the driver valve in the device of the invention are described in more detail below with reference to FIG. 6. The constructions of the inlet valve and the outlet valve can be the same as the construction of the driver valve or can be different. In particular, it is envisaged that the inlet valve or the outlet valve or each of them need not be constructed so that a quantity of a primary fluid can be retained in an associated void.

The discharge valve wall 108 can include a discharge valve which makes use of an electro-osmotic effect. The valve can operate functionally in the same way as the valve which is described above with reference to FIG. 3, in which the flow channel 162 extends between the reservoir for the primary fluid between the membrane 112 and the outer wall 102 of the housing, and the inlet to the hollow rod 110. The flow channel which connects the reservoir and the hollow rod can be provided, at least along part of its length, by a compressible tube, which can be closed against flow of fluid by the action on it of a valve fluid which can be pressurised by means of an electro-osmotic device 168, 170. These components of the discharge valve should preferably be fitted entirely within the discharge valve end wall.

The base includes a power source in the form of a battery which can be used to power the valves as they operate between their open and closed positions.

The sequence of operation of the valves during discharge of primary fluid from the device involves:

1. Open inlet valve 114.

2. Open driver valve 116 to withdraw primary fluid from the reservoir into a holding void which is associated with the driver valve.

3. Close inlet valve 114.

4. Open outlet valve 118.

5. Close driver valve 116 to expel the primary fluid from the holding void which is associated with the driver valve.

FIG. 6 shows a valve which can be used as the driver valve 116, and possibly also as the inlet valve or the outlet valve or each of them, in a device as shown in FIGS. 4 and 5. The valve 116 comprises a core 204 in the form of a stainless steel rod. The diameter of the core is 2 mm. The core comprises an inlet section 206, a valve region 208 and an outlet section 210. The rod has a plurality of grooves extending along its length.

The valve includes a tube 214 of an elastomeric material which is a tight fit around the core to close the grooves along their lengths so that the grooves in the external surface of the rod become channels. The tube is formed from a silicone rubber, with a wall thickness of about 0.2 mm.

In the valve region 208, the core 204 has an annular recess 215 formed in it. The cross-sectional area of the core decreases through the valve region from the inlet section 206 towards the outlet section to a point 207 at which the cross-sectional area of the core is at a minimum, and then increases towards the outlet section. The ratio of the length of the portion of the recess from the inlet section to the point of minimum cross-sectional area to the length of the portion from the point of minimum cross-sectional area to the outlet section is about 7.

The core 204 and the surrounding tube 214 are located within a housing 216. In the inlet and outlet sections 206, 210, the core and the surrounding tube are a tight fit in the housing so that the housing supports the tube against outward expansion due to the pressure of fluid within the grooves in the core.

Tight annular seals 217 are provided around the surrounding tube 214 between the tube and the housing 216 at opposite ends of the valve region, defining a void 218 surrounding the core in the valve region between the inlet and outlet sections.

On one side of the core, the void 218 is in fluid communication with an electro-osmotic device. The device comprises a laminate 220 of a layer of a porous dielectric material which consists of a silica, sandwiched between a pair of electrodes.

The laminate separates the void 218 from a reservoir 222 for a displacement fluid.

In use, the primary fluid flows along the grooves in the inlet section 206 of the core 204. The primary fluid is able to flow into the recess 215 which surrounds the core in the valve region 208, while there is a space between the core and the internal surface of the surrounding tube 214 throughout the length of the valve region. The primary fluid is then able to flow from the valve region and out of the valve through grooves in the outlet section 210 of the core. However, when the surrounding tube contacts the core in the valve region continuously around the periphery of the core, the tube presents an obstacle to flow of the primary fluid so that the valve becomes closed.

The space between the core 204 in the valve region 208 and the surrounding tube 214 is controlled by movement of displacement fluid between the reservoir 222 and the void 218. Movement of the displacement fluid from the reservoir 222 into the void 218 causes the tube 214 to be forced towards the core 204, resulting in a reduction in the distance between the core and the tube, and ultimately to contact between the tube and the core continuously around the periphery of the core so that the path for flow of the primary fluid through the valve becomes closed.

The shape of the core 204 in the valve region 208, involving a gradual reduction in diameter along its length from the inlet section 206 toward a point 207 where the diameter is at a minimum, as discussed above, means that the tube 214 tends to contact the core first at the end of the valve region 208 which is closest to the inlet section 206, and then progressively to contact the core along the length of the valve region towards the outlet section 210. This results in progressive expulsion of the primary fluid from the valve region of the pump as a result of pumping displacement fluid from the reservoir 222 into the void 218 around the tube 214 in the valve region of the device.

1. A delivery device for a primary fluid, which comprises:
a. a tubular housing which comprises a reservoir for the primary fluid, and a chamber for a displacement fluid which extends coaxially with the reservoir inside the housing and is separated from the reservoir by means of a membrane,
b. an outlet from the reservoir for the primary fluid,
c. a device for driving the displacement fluid under pressure into the chamber so that the membrane is deformed into the reservoir to cause primary fluid in the reservoir to be displaced towards the outlet.

2. A delivery device as claimed in claim 1, in which the chamber is located within the reservoir.

3. A delivery device as claimed in claim 1, in which the reservoir is located within the chamber.

4. A delivery device as claimed in claim 1, which includes a source for the displacement fluid

5. A delivery device as claimed in claim 4, in which the source of the displacement fluid is located within the housing.

6. A delivery device as claimed in claim 5, in which the source of the displacement fluid is provided by a source reservoir which extends coaxially within the housing with the chamber and the reservoir for the primary fluid.

7. A delivery device as claimed in claim 1, in which the driver device is located at one end of the housing.

8. A delivery device as claimed in claim 1, in which the driver device comprises an electro-osmotic device.

9. A delivery device as claimed in claim 1, which includes an outlet valve to control flow of the primary fluid through the reservoir outlet.

10. A delivery device as claimed in claim 1, in which the cross-section of the housing when viewed along its axis is approximately circular.

11. A delivery device as claimed in claim 1, in which the housing is elongate.

12. A delivery device as claimed in claim 1, in which the cross-section of the housing when viewed along its axis is approximately circular, and in which the ratio of the length of the housing measured along its axis to its diameter is at least about 1.0.

13. A delivery device as claimed in claim 1, in which the material of the membrane is resiliently deformable.

14. A delivery device as claimed in claim 1, in which the membrane is folded, so that it can be deformed into the reservoir by unfolding.

15. A delivery device as claimed in claim 1, in which the reservoir and the membrane are constructed so that, when exposed to pressurised displacement fluid, the membrane first contacts the reservoir wall in an inlet region
